(19) United States
(12) Patent Application Publication
(10) Pub. No.: US 2005/0031627 A1
(43) Pub. Date: Feb. 10, 2005

(54) Methods for preparing immunoconjugates

(76) Inventors: Gregory J Mazzola, King of Prussia, PA (US); William K. Wang, Cambridge, MA (US); Gerardo A. Zapata, Freemont, CA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/500,533

(22) PCT Filed: Jan. 2, 2003
(86) PCT No.: PCT/US03/00205

(30) Foreign Application Priority Data
Jan. 3, 2002 (US) 60345305

(51) Int. Cl. A61K 39/395; C07K 16/46

(52) U.S. Cl. 424/178.1; 530/391.1; 540/462

(57) ABSTRACT

Improved methods for preparing immunoconjugates are disclosed.

### FIELD OF THE INVENTION

This invention relates to methods for the preparation of immunoconjugates, in particular maytansinoids conjugated to a monoclonal antibody.

### BACKGROUND OF THE INVENTION

Tumor-activated pro-drugs having a highly cytotoxic maytansinoid conjugated to a cell-binding agent such as a monoclonal antibody have been described in U.S. Pat. No. 5,208,020. The antibody is directed to a tumor-specific antigen and delivers the maytansinoid directly to the tumor site. In conjugate form, the maytansinoid is inactive and can be administered without causing systemic toxicity to a patient. After binding to the surface of a tumor cell, the conjugate is internalized and the maytansinoid is released from the antibody and can exert its cytotoxic effect on the tumor cell.

The maytansinoids are anti-mitotic drugs 100 to 1000-fold more cytotoxic than conventional cancer chemotherapeutic agents such as methotrexate, daunorubicin and vincristine. If administered in unconjugated form, maytansinoids can cause adverse effects to the central nervous system and gastrointestinal tract. In conjugated form, it has been recognized that the full cytotoxic potential of the maytansinoids can be observed only if the maytansinoids can be released in unmodified form at the tumor target site.

Preparation of maytansanoid derivatives with cleavable but highly stable linkers for conjugating maytansinoids such as maytansinol and the N-methyl-L-alanine maytansinoid derivative DM1 to monoclonal antibodies have been disclosed in U.S. Pat. No. 5,208,020. The synthesis of disulfide- and thiol-containing maytansinoids which can be linked to cell-binding agents via a disulfide or any other sulfur-containing link such as thioether or thioester links is disclosed in U.S. Pat. No. 5,416,064.

In general, antibody-cytotoxin conjugates are prepared by a multi-step process. First, antibody is covalently attached to a linker in a modification reaction and unreacted components and reaction products separated from antibody-linker conjugate by desalting. Next, purified antibody-linker conjugate is reacted with modified cytotoxin to form the antibody-cytotoxin conjugate. The conjugate is purified from unreacted components, solvent and reaction products by size exclusion chromatography. See, e.g., Chari et al. in Cancer Research 52, 127-131 (1992) and Liu et al. in Proc. Natl. Acad. Sci. (USA) 93, 8618-8623 (1996).

While the modification and conjugation reactions of these processes can be used on a small scale, the purification techniques introduce limitations in large-scale production operations, particularly because of their low efficiency, yield and productivity. Thus, a need exists to have alternative procedures available which are scalable and offer improvements in efficiency, yield and productivity resulting in lower drug product manufacturing costs.

### SUMMARY OF THE INVENTION

One aspect of the present invention is a method for conjugating a maytansinoid to an antibody comprising the steps of:
a. reacting a disulfide-containing linker with the antibody at about pH 5.0 to about pH 8.0 to form a modified antibody;
b. removing unreacted linker from the modified antibody by tangential flow filtration;
c. conjugating the modified antibody with the maytansinoid at about pH 6.0 to about pH 6.5 in a solvent comprising dimethylacetamide and/or acetonitrile; and
d. purifying the modified antibody-maytansinoid conjugate by ion exchange chromatography or with SP-Sepharose.

### BRIEF DESCRIPTION OF THE DRAWINGS

FIG. 1 provides N-succinimidyl 4-(2-pyridyldithio) pentanoate (SPP):Monoclonal Antibody (Mab) ratios for each experiment performed in Example 2 at thirty minute-intervals.

### DETAILED DESCRIPTION OF THE INVENTION

All publications, including but not limited to patents and patent applications, cited in this specification are herein incorporated by reference as though fully set forth.

The term “antibody” as used herein and in the claims includes antibodies of all immunoglobulin types, such as IgG, IgA, IgM, IgD and IgE, and fragments thereof, and includes antibodies and antibody fragments of all origins, such as polyclonal antibodies, monoclonal antibodies, humanized antibodies and human antibodies produced in transgenic animals or transgenic animal cell culture.

“Antibody fragment” as used herein and in the claims defines a portion of an intact antibody comprising the antigen-binding site or variable region of the intact antibody. Examples of antibody fragments include Fab, Fab′, Fab′-SH, F(ab)₂ and Fv fragments, diabodies, single-chain Fv molecules and single-chain molecules comprising light chain or heavy chain variable domains or light chain or heavy chain complementarity determining regions (CDRs).

Methods are provided for preparing antibody-maytansinoid conjugates comprising the steps of reacting a disulfide-containing linker with the antibody to form a modified antibody; removing unreacted linker from the modified antibody by tangential flow filtration (TFF); conjugating the modified antibody with a maytansinoid; and purifying the antibody-maytansinoid conjugate. Preferably, the maytansinoid is DM1, shown in Formula I. The antibody-maytansinoid conjugate prepared by the method of the invention can subsequently be formulated to provide bulk drug substance.

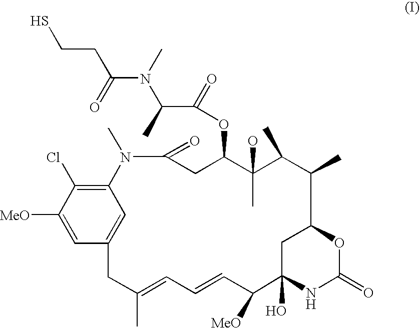

The modified antibody is produced by supplying a desired monoclonal antibody (Mab) at a concentration of about 25 g/L in a buffer at about pH 5.0 to about pH 8.0. Preferred buffer species are citric acid, succinic acid, 2-(N-morpholino)ethanesulfonic acid (MES), piperazine-N-N′-bis(2-ethanesulfonic) acid (PIPES), imidazole, 3-(N-morpholino)propanesulfonic acid (MOPS) and phosphoric acid. The antibody is then reacted with a disulfide-containing linker to yield a linker-modified antibody. Preferably, the reaction is incubated for at least 120 min at ambient temperature with continuous mixing. A preferred linker species is N-succinimidyl 4-(2-pyridyldithio) pentanoate (SPP), shown in Formula II.

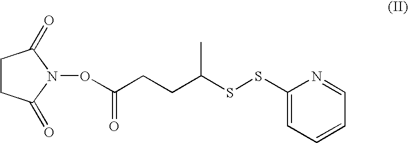

A tangential flow filtration (TFF) step is used to remove unreacted linker and ethanol from the linker-modified antibody. Subsequent diafiltration is used to filter the linker-modified antibody into the conjugation reaction buffer and adjust the concentration. Preferably, the ultrafiltration membrane is a Millipore Biomax 50® polyethersulfone cartridge. The conjugation reaction buffer is at about pH 6.0 to about pH 6.5 and contains a chelating agent, preferably EDTA. Preferred diafiltration parameters are a total membrane surface area of about 2.5 m², an operational transmembrane pressure (TMP) of about 16 psi to about 35 psi and an operating temperature of about 2° C. to about 25° C.

The TFF and diafiltration steps result in efficient reduction in the amounts of the unreacted linker and ethanol with control of the product concentration at all points in the process. The result is a more scalable, controlled, and productive process compared to a size exclusion chromatography liquid chromatography step.

A maytansinoid such as DM1 is prepared in dimethylacetamide (DMA), preferably 10 mM DM1, and mixed with the linker-modified antibody for the conjugation reaction. Preferably, the reagents are reacted for about 20 hrs at ambient temperature with continuous mixing. Additionally, the maytansinoid may be prepared in a solvent containing acetonitrile.

Antibody-DM1 conjugate is purified from unreacted DM1, DMA and aggregates by liquid chromatography on an ion exchange column. Preferably, the column is ceramic hydroxyapatite. Preferably, the column is pre-equilibrated and equilibrated with pH 6.5 buffer, loaded at a load ratio of 10-15 g/L, washed with equilibration buffer and eluted with pH 6.5 buffer containing NaCl. In a preferred embodiment, the pre-equilibration buffer is 400 mM sodium phosphate, pH 6.5 and the equilibration buffer is 30 mM sodium phosphate, 70 mM NaCl, pH 6.5, the wash buffer is 30 mM sodium phosphate, 70 mM NaCl, pH 6.5 and the elution buffer is 30 mM sodium phosophate, 300 mM NaCl, pH 6.5 and the column flow rate is 300 cm/hr.

The ceramic hydroxyapatite column (Macro-Prep Ceramic Hyroxyapatite, Type I from Bio-Rad Laboratories) efficiently reduces the amounts of product aggregate, DMA and unreacted DM1 from the conjugated antibody product. It is also possible to use ion exchange liquid chromatography media to achieve the same objectives. Ion exchange chromatography media are cation exchangers (e.g., SP-Sepharose Fast Flow and CM-Sepharose Fast Flow, both from Amersham Pharmacia Biotech) or anion exchangers (e.g., Q-Sepharose Fast Flow from Amersham Pharmacia Biotech, Macro-Prep DEAE Support from Bio-Rad Laboratories).

This step results in efficient buffer exchange into the formulation buffer with control of the product concentration at all points in the process. The result is a more scalable, controlled and productive process compared to a size exclusion chromatography liquid chromatography step.

The present invention will now be described with reference to the following specific, non-limiting examples. In all ratios presented in the examples, the number presented in the text is presumed to be the first number in the ratio and the second number is presumed to be 1 and is not presented in the text. For example, a ratio of 3:1 is presented as 3.

### EXAMPLE 1

### Conjugation of DM1 with Anti-Human CanAg Monoclonal Antibody

A disulfide-containing linker of formula II was attached to an anti-human CanAg monoclonal antibody containing the CDRs of C242 (See U.S. Pat. No. 5,552,293) by the following method. A 26.6 g/L antibody solution was supplied in a pH 6.0 buffer and the monoclonal antibody solution was adjusted to pH 6.5 with 0.5 M NaOH. A 10 mM linker stock was made in ethanol and the actual linker concentration was determined to be 9.7 mM. The reagents were combined in the reaction vessel with continuous mixing in the following order: 75.2 mL monoclonal antibody, 174.8 mL pH 6.5 buffer, 4.3 mL ethanol, and 8.8 mL linker solution. The solution was mixed five minutes before the linker solution was added. The final reaction contained 7.6 mg/mL monoclonal antibody, 5% ethanol and 0.33 mM linker. The reaction was incubated for 150 min at 25° C. (room temperature) with continuous mixing throughout the reaction incubation.

Tangential flow ultrafiltration and diafiltration steps were used to reduce the amounts of unreacted linker and ethanol in the product solution, to diafilter the modified antibody into the conjugation reaction buffer and to adjust the product concentration. The ultrafiltration membrane was a Millipore Biomax 50® polyethersulfone cartridge. The conjugation reaction buffer was a pH 6.5 buffer containing EDTA. The tangential flow ultrafiltration system was rinsed with water for injection (WFI) and equilibrated with the conjugation reaction buffer before the product solution was introduced. The product was diafiltered with five diavolumes of the pH 6.5 conjugation reaction buffer. The product was overconcentrated to maximize yield by rinsing the filtration system and adding the rinse to the final product. The final product concentration was determined to be 7.4 mg/mL. Parameters for this operation were a total membrane surface area of 0.1 m², an operational TMP of 10 psi, and an operating temperature of 25° C.

The DM1 drug was then attached to the linker-modified monoclonal antibody. A 10 mM DM1 stock was made in dimethylacetamide (DMA). The DM1 concentration was determined by a spectrophotometric assay to be 0.011 M. The reagents were combined in the reaction vessel with continuous mixing in the following order: 210 mL linker modified monoclonal antibody, 435 mL pH 6.5 conjugation reaction buffer, 11.6 mL DMA, and 8.4 mL DM1 solution. The solution was mixed five minutes before the final DM1 solution was added. The final reaction contained 2.35 mg/mL monoclonal antibody, 3% DMA and 0.14 mM DM1. The reaction proceeded for 23 hr at 22° C. (room temperature) with continuous mixing throughout the reaction incubation.

A ceramic hydroxyapatite liquid chromatography column was used to purify the antibody-DM1 conjugate from unreacted DM1, DMA and aggregates. The entire conjugation reaction mixture was processed in a single column injection. The conjugation reaction mixture was filtered using a 0.22 micron filter prior to loading. The 106 mL column was equilibrated with 636 mL pH 6.5 buffer, loaded at load ratio of 14.8 g/L and washed with 318 mL pH 6.5 buffer. After the product was eluted, the column was cleaned with 0.5 M NaOH and stored in 0.01 M NaOH. The column flow rate was 300 cm/hr. The antibody-DM1 conjugate was collected from the column as a single peak measured by UV absorbance. The total eluate volume collected for further processing was 379 mL with a conjugate concentration of 3.6 g/L. The DM1:antibody ratio was determined to be 3.2 by spectrophotometric assay.

A tangential flow ultrafiltration and diafiltration step was used to diafilter the antibody-DM1 conjugate into the bulk formulation buffer and to adjust the product concentration. The ultrafiltration membrane was a Millipore Biomax 50® polyethersulfone cartridge. The final formulation buffer was at pH 6.0. The filtration system was rinsed with WFI and equilibrated with the formulation buffer before the product solution was introduced. The product was concentrated to a target 10 mg/mL and buffer exchanged with eight diavolumes. When diafiltration was complete, the product was overconcentrated to maximize yield by rinsing the filtration system and adding the rinse to the final product. The final product concentration was determined to be 9.61 mg/mL. Parameters for this operation were a total membrane surface area of 0.1 m², an operational TMP of 10 psi, and an operating temperature of 22° C.

The present invention may be embodied in other specific forms without departing from the spirit or essential attributes thereof, and, accordingly, reference should be made to the appended claims, rather than to the foregoing specification, as indicating the scope of the invention.

The following examples are intended to exemplify but not limit the several embodiments of the invention.

### EXAMPLE 2

### Antibody Attachment to SPP Linker: Conditions Leading to Different SPP:Mab Ratios

In order to investigate conditions necessary to control the ratio of linker to monoclonal antibody (Mab) attachment, a factorial experiment was designed that varied pH, Mab concentration and linker concentration in statistical combinations.

In a multi-variable full factorial experiment, reaction mixtures were prepared to study statistical combinations of the variables for Mab concentration, SPP molar excess, and pH as summarized in Table 1.

TABLE 1

| Factor | Low Level | Midpoint | High Level |
|---|---|---|---|
| Mab Conc (mg/mL) | 3.8 | 7.6 | 11.4 |
| Linker Molar Excess | 3 | 6.3 | 9.6 |
| pH | 5 | 6.5 | 8 |

| | |
|---|---|
| Metrics: | SPP:Mab ratio at 30 min. intervals for 3 |
| | hours |

Each of 10 reaction mixtures (experiments) was prepared according to Table 2.

TABLE 2
[SPP] in SPP Stock: 17.9 mM

| | | Experiment No. | | | | | | | | | |
|---|---|---|---|---|---|---|---|---|---|---|---|
| | | 1 | 2 | 3 | 4 | 5 | 6 | 7 | 8 | 9 | 10 |
| Scale | mg | 14 | 20 | 7 | 7 | 14 | 7 | 20 | 7 | 20 | 20 |
| Starting Protein | mg/mL | 24.9 | 24.9 | 24.9 | 24.9 | 24.9 | 24.9 | 24.9 | 24.9 | 24.9 | 24.9 |
| conc. | | | | | | | | | | | |
| Molar Excess of | ratio | 6.3 | 9.6 | 9.6 | 3 | 6.3 | 9.6 | 3 | 3 | 9.6 | 3 |
| SPP | | | | | | | | | | | |
| [EtOH] | % | 5.0 | 5.0 | 5.0 | 5.0 | 5.0 | 5.0 | 5.0 | 5.0 | 5.0 | 5.0 |
| RXN [Mab] | mg/mL | 7.6 | 11.4 | 3.8 | 3.8 | 7.6 | 3.8 | 11.4 | 3.8 | 11.4 | 11.4 |
| Reagents to Add: | | | | | | | | | | | |
| Mab (underivatized) | mL | 0.562 | 0.803 | 0.281 | 0.281 | 0.562 | 0.281 | 0.803 | 0.281 | 0.803 | 0.803 |
| Buffer pH | | 6.5 | 5.0 | 5.0 | 5.0 | 6.5 | 8.0 | 5.0 | 8.0 | 8.0 | 8.0 |
| Volume of Buffer to | mL | 1.188 | 0.863 | 1.469 | 1.469 | 1.188 | 1.469 | 0.863 | 1.469 | 0.863 | 0.863 |
| add | | | | | | | | | | | |
| EtOH | μL | 59 | 15 | 67 | 84 | 59 | 67 | 65 | 84 | 15 | 65 |
| SPP in EtOH | μL | 34 | 73 | 26 | 8 | 34 | 26 | 23 | 8 | 73 | 23 |
| Total Reaction | mL | 1.84 | 1.75 | 1.84 | 1.84 | 1.84 | 1.84 | 1.75 | 1.84 | 1.75 | 1.75 |
| Volume | | | | | | | | | | | |
| Final Mab conc. | | 7.6 | 11.4 | 3.8 | 3.8 | 7.6 | 3.8 | 11.4 | 3.8 | 11.4 | 11.4 |

Buffers
50 mM Sodium Acetate, pH 5.0
50 mM Succinate, pH 6.5
50 mM Tris Base, pH 8.0

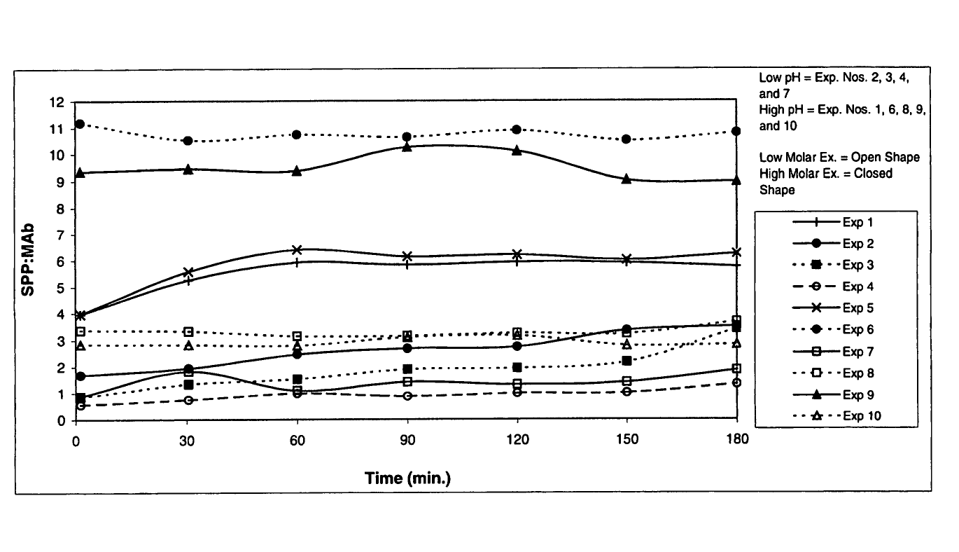

For the linker, a 20 mM linker stock was prepared in ethanol (with the actual concentration determined to be 17.9 mM by spectrophotometric assay) and used for all reactions. Linker reactions were carried out in 50 mM sodium acetate for pH 5.0; 50 mM; succinate for pH 6.5; and 50 mM Tris base for pH 8.0. Each reaction contained the prescribed amount of Mab and 5% v/v ethanol. Each reaction was allowed to continue for 180 minutes, with samples drawn at 30 minute intervals. The samples were buffer exchanged into 50 mM succinate, 2 mM EDTA, pH 6.5 using centrifugal membrane filters to remove unreacted linker. The samples were then analyzed by spectrophotometric assay for linker and antibody concentrations. Final SPP:Mab ratios varied from 1 to 11. FIG. 1 shows the time dependence for each reaction mixture and the resulting SPP:Mab ratio.

These results demonstrate that by varying the molar excess of linker, the pH, the time the reaction is allowed to proceed, and the Mab concentration in the correct combinations, SPP:Mab ratios may be manipulated to the desired level.

This procedure was repeated at a higher scale using 200 mg of monoclonal antibody per reaction, and buffer exchanged using size exclusion chromatography. The resulting SPP:Mab ratios again ranged from 1 to 11.

The changes observed in the SPP:Mab ratio demonstrate the ability to alter the ratio as needed or desired. The SPP:Mab ratio directly effects the amount of DM1 conjugation.

Additionally, separate linker reactions were performed using antibody dialyzed into three buffers (sodium acetate, sodium phosphate, and succinate) at three different concentrations (20 mM, 50 mM, and 100 mM) and three different pHs (5.5, 6.0, and 6.5). Final linker to antibody ratios ranged from 3.4 to 4.9. The linked antibody from the pH 6.5 experiments were taken through the DM1 conjugation process to yield DM1 to antibody ratios of ≧3.3. These results demonstrate that pH and buffer type in the linker reaction have little effect on the resulting SPP:Mab ratio.

### EXAMPLE 3

### Antibody Attachment to Linker Using Ethanol or Acetonitrile as the Solvent in the Reaction Mixture

Antibody stock solution at 27 mg/mL concentration was adjusted to pH 6.5 with 0.5 M NaOH. Various amounts of ethanol were added to bring the final amount of ethanol to concentrations ranging from 3.5% to 12.5% v/v. A 10 mM SPP stock was prepared in ethanol with the actual concentration determined to be 9.6 mM by spectrophotometric assay. The reaction buffer was 50 mM succinate, pH 6.5. Final protein concentration was 7.6 mg/mL in all cases. The reaction mixture was buffer exchanged by size exclusion chromatography after 120 minutes and was assayed spectrophotometrically to ascertain the SPP:Mab ratio. No significant differences were observed, with the SPP:Mab ratios ranging from 4.2 to 4.8. This example demonstrates that the final ethanol concentration in the SPP reaction can range from 3.5 to 12.5% v/v.

Antibody stock solution at 14.4 mg/mL concentration was adjusted to pH 6.5 with 0.5 M NaOH. Various volumes of acetonitrile were added to bring the final acetonitrile to concentrations ranging from 3.8% to 15% in the final reaction mixture. A 10 mM SPP stock was prepared in acetonitrile with the actual concentration determined to be 8.6 mM by spectrophotometric assay. The reaction buffer was 50 mM succinate, pH 6.5. Final protein concentration was 7.6 mg/mL in all cases. The reaction mixtures were buffer exchanged by size exclusion chromatography after 120 minutes and were assayed spectrophotometrically to ascertain the SPP:Mab ratio. The SPP:Mab ratios ranged from 5.0 to 6.5. This example demonstrates that at a pH of 6.5, acetonitrile may be used instead of ethanol with similar or greater levels of SPP attachment achieved compared to ethanol.

### EXAMPLE 4

### Additional Reaction Time Study of Antibody Attachment to Linker

Antibody stock at 27 mg/mL concentration was adjusted to pH 6.5 with 0.5 M NaOH. Ethanol was added to bring the final concentration to 5% v/v. A 10 mM SPP stock was prepared in ethanol with the actual concentration determined to be 9.6 mM by spectrophotometric assay. The reaction buffer was 50 mM succinate, pH 6.5. Final protein concentration was 7.6 mg/mL in all cases. The linker attachment reaction kinetics were studied by taking samples at 10 minute intervals for the first 30 minutes, then at 30 minute intervals up to 180 minutes. The samples taken were then buffer exchanged by size exclusion chromatography (Sephadex G-25) and assayed spectrophotometrically to ascertain the SPP:Mab ratio. The SPP:Mab ratios ranged from 1.0 to 5.9. This example demonstrates that the time of the reaction can be utilized to vary the level of SPP attachment to the Mab. This variation in SPP attachment allows different levels of DM1 conjugation in the subsequent reaction, since the amount of SPP attached to the Mab will directly effect the final level of DM1 conjugation.

### EXAMPLE 5

### Antibody Attachment to Linker Using Various Antibody Starting Concentrations

Antibody stock at 27 mg/mL concentration was adjusted to pH 6.5 with 0.5 M NaOH. Various amounts of the antibody stock solution were added in separate reaction mixtures to final concentrations ranging from 3.8 mg/mL to 25.1 mg/mL. A 10 mM SPP stock was prepared in ethanol with the actual concentration determined to be 9.6 mM by spectrophotometric assay. The reaction buffer was 50 mM succinate, pH 6.5. The reaction mixture was buffer exchanged by size exclusion chromatography after 120 minutes and was assayed spectrophotometrically to ascertain the SPP:Mab ratio. The SPP:Mab ratios ranged from 4.1 to 6.3. This example demonstrates that the SPP reaction at pH 6.5 is not effected to a large extent by a protein concentration in the 3.8-25.1 mg/mL range, allowing operational flexibility with respect to protein concentration.

### EXAMPLE 6

### Conjugation of the Modified Antibody with DM1 in the Presence of Acetonitrile

A conjugation reaction was performed in which acetonitrile (ACN) replaced DMA as the solvent in the reaction mixture. A 10 mM DM1 stock solution was prepared in neat acetonitrile, and the concentration determined by spectrophotometric assay. The modified antibody used in the reaction had a SPP:Mab ratio of 5.1. A molar excess of DM1 of 1.5 (relative to SPP modified Mab) was utilized in the reaction. The reaction buffer was 50 mM succinate, 2 mM EDTA, pH 6.5; the reaction final mixture contained 5% v/v ACN. The reaction was allowed to proceed for 22 hours, at which point the reaction mixture was purified over a G-25 column. The resulting DM1:Mab ratio was 2.4 as determined by spectrophotometric assay. This examples demonstrates that ACN can be used in place of ethanol as the solvent component.

An additional experiment was performed in which ACN and DMA based conjugation reactions were compared head-to-head. Each reaction was run for five hours and used a 1.7 molar excess of DM1 to SPP-modified Mab. Each reaction was purified over a G-25 column. The DMl:Mab ratios were 1.2 and 1.5 for the ACN and DMA reactions, respectively. These experiments demonstrate that the solvent used had little or no effect on the resulting conjugation efficiency.

### EXAMPLE 7

### Purification of the DM1-Antibody Conjugate Using SP-Sepharose FF

An SP-Sepharose fast flow column (from Amersham Pharmacia Biotech) was equilibrated with a 30 mM Phosphate buffer at pH 6.5. Unpurified conjugation reaction mixture was loaded onto the column at a load ratio of 15 mg/mL of resin. After loading, the column was washed with equilibration buffer and then eluted with a 30 mM sodium Phosphate buffer, pH 6.5 containing 70 mM Sodium Chloride (NaCl). A non-bound, eluate, and eluate tail fraction was collected. All fractions, including the load material, were assayed using a Size-Exclusion Chromatography (SEC-HPLC) method for determining aggregate content. The unpurified load material had an aggregate content of 2.3%. The eluate fraction contained a product peak and had an aggregate content of 1.77%.

1. A method for conjugating a maytansinoid to an antibody comprising the steps of:
a. reacting a disulfide-containing linker with the antibody at about pH 5.0 to about pH 8.0 to form a modified antibody;
b. removing unreacted linker from the modified antibody by tangential flow filtration;
c. conjugating the modified antibody with the maytansinoid at about pH 6.0 to about pH 6.5 in a solvent comprising dimethylacetamide; and
d. purifying the modified antibody-maytansinoid conjugate by ion exchange chromatography.

2. A method for conjugating a maytansinoid to an antibody comprising the steps of:
a. reacting a disulfide-containing linker with the antibody at about pH 5.0 to about pH 8.0 to form a modified antibody;
b. removing unreacted linker from the modified antibody by tangential flow filtration;
c. conjugating the modified antibody with the maytansinoid at about pH 6.0 to about pH 6.5 in a solvent comprising acetonitrile; and
d. purifying the modified antibody-maytansinoid conjugate by ion exchange chromatography.

3. The method of claim 1 where the maytansinoid is DM1.

4. The method of claim 1 where the linker is SPP.

5. The method of claim 1 where the maytansinoid is DM1 and the linker is SPP.

6. The method of claim 1 further comprising the step of:
e. formulating the modified antibody-maytansinoid conjugate.

7. The method of claim 1 wherein the ion exchange chromatography is performed on a ceramic hydroxyapatite column.

8. The method of claim 1 wherein the ion exchange chromatography is performed on SP-Sepharose.

9. An antibody-maytansinoid conjugate prepared by the method of claim 1.

10. The method of claim 2 where the maytansinoid is DM1.

11. The method of claim 2 where the linker is SPP.

12. The method of claim 2 where the maytansinoid is DM1 and the linker is SPP.

13. The method of claim 2 further comprising the step of:
e. formulating the modified antibody-maytansinoid conjugate.

14. The method of claim 2 wherein the ion exchange chromatography is performed on a ceramic hydroxyapatite column.

15. The method of claim 2 wherein the ion exchange chromatography is performed on SP-Sepharose.

16. An antibody-maytansinoid conjugate prepared by the method of claim 2.
